(19) United States
(12) Patent Application Publication
(10) Pub. No.: US 2018/0002590 A1
(43) Pub. Date: Jan. 4, 2018

(54) IN SITU SWELLING OF WATER-SWELLABLE POLYMERS DOWNHOLE

(71) Applicant: Halliburton Energy Services, Inc., Houston, TX (US)

(72) Inventors: Venkata Satya Srikalyan BHAMIDIPATI, Kingwood, TX (US); Humberto Almeida OLIVEIRA, Lagoa Santa (BR); Zheng LU, Kingwood, TX (US)

(21) Appl. No.: 15/545,203

(22) PCT Filed: Feb. 25, 2015
(86) PCT No.: PCT/US2015/017452

§ 371 Date: Jul. 20, 2017

(51) Int. Cl. C09K 8/24; C09K 8/36; C09K 8/80; C07C 43/13

(52) U.S. Cl. CPC C09K 8/24; C09K 8/80; C09K 8/36; C07C 43/13; C09K 2208/26

(57) ABSTRACT

Invert emulsions may be used in downhole operations to delay the swelling of water-swellable polymers. For example, a treatment fluid may be introduced into a wellbore penetrating a subterranean formation, the treatment fluid comprising an emulsion with an continuous oil phase and a discontinuous aqueous phase, an emulsifier, and a water-swellable polymer suspended in the continuous oil phase, wherein the aqueous discontinuous phase has a pH of about 0 to about 11; the emulsion may be broken while the treatment fluid in a portion of the subterranean formation; and the water-swellable polymer may be swollen into a swollen polymer, thereby reducing fluid flow through the portion of the subterranean formation. In some instances, for carbonate subterranean formation, the aqueous discontinuous phase may have a pH of about 7 to about 11.

### BACKGROUND

The present disclosure relates to the use of water-swellable polymers in downhole operations.

Wellbore fluids used in oil and gas exploration and production use a variety of additives to achieve a desired property for the fluid or to produce a desired result in the wellbore. One example of an additive that can serve many purposes is a water-swellable material. For example, in a swollen form, these materials can increase the solid/liquid volume ratio of a wellbore fluid, which when placed in a permeable portion of the formation, may allow for the swollen materials to plug or reduce fluid flow through that permeable portion of the formation resulting in problems such as lost circulation of wellbore treatment fluids.

Generally, the water-swellable polymers are placed downhole at the permeable zone by mixing with a carrier fluid and introducing the fluid downhole. However, such techniques may, in some instances, limit the concentration of the water-swellable polymers in the carrier fluid because as the polymer swells, the fluidity or pumpability of the fluid decreases. Because many water-swellable polymers can increase in volume by about 400%, the pumpability of the fluid may decrease prematurely even with relatively low concentrations of water-swellable polymer and interfere with placement in the correct location. This may, in some cases, reduce the depth of placement to near-wellbore locations (e.g., less than about 50 ft from the wellbore). To mitigate significant viscosity increases, large quantities of carrier fluid may be used to facilitate pumping, which can be time consuming and costly.

In cases, where placement deep inside a fracture or cavernous zones becomes problematic due to swollen particle sizes, carrier fluids with high salt concentrations are used in which the particle swelling is less. Once the partially swollen particles are placed within a zone as desired, a subsequent fluid based on fresh water or low-salt concentration brine, is pumped through the swollen particle mass to increase the swollen particle volume in situ. The decrease in swollen volume of water-swellable polymers by salt solutions is dependent on the type of salt and concentration. It is a common practice to use monovalent salts such as sodium chloride or potassium chloride. The swellable-particle volume increase with monovalent salts is still significantly high, and may be subject to the same limitations as fresh water systems.

The use of fluids containing divalent ions further decreases the swelling of the particles, which allows for ease of pumping and deeper placement of particles inside a high permeability zone, such as a fracture. However, further swelling of the particles in situ upon pumping fresh water or an aqueous fluid containing lower salt concentration is negligible. Therefore, plugging of such zones by water-swellable polymers is not practical. Thus, there is a need for fluid compositions containing water-swellable polymers that allow for less swelling during placement and enhanced swelling in situ after placement.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 shows an illustrative schematic of a system that can deliver treatment fluids of the present disclosure to a downhole location, according to one or more embodiments.

FIGS. 2A-B show a water-swellable polymer after inclusion in a control seawater sample and an invert emulsion sample according to at least one embodiment described herein, respectively.

### DETAILED DESCRIPTION

The present disclosure relates to the use of water-swellable polymers in downhole operations. More specifically, the embodiments described herein utilize invert emulsions to delay the swelling of water-swellable polymers. As used herein, the term “water-swellable” refers to the ability of the material to increase its volume and/or mass when in contact with an aqueous fluid.

Generally, delayed swelling is achieved by suspending the water-swellable polymer in the continuous oil phase of the invert emulsion. Then, when the emulsion is broken the discontinuous aqueous phase causes the water-swellable polymer to swell. This swollen polymer may then reduce fluid flow (i.e., the amount of fluid flowing) through the subterranean formation where the emulsion was broken and the water-swellable polymer was swollen.

In some instances, the discontinuous aqueous phase of the emulsion may be neutral to basic (e.g., about pH 7 to about pH 11). This may advantageously allow for the implementation of the treatment fluids and methods described herein in carbonate subterranean formations where acidic fluids cause formation damage. As used herein, the term “carbonate subterranean formation” refers to a subterranean formation are substantially (i.e., at least 50%) inorganic carbonate material. Examples of such inorganic carbonate material may include, but are not limited to, limestone and dolomite.

In some embodiments, treatment fluids described herein may be invert emulsions comprising a continuous oil phase, a discontinuous aqueous phase with a pH of about 0 to about 11, an emulsifier, and a water-swellable polymer that is suspended in the continuous oil phase.

Examples of oils suitable for use as the continuous oil phase may include, but are not limited to, alkanes, olefins, aromatic organic compounds, cyclic alkanes, paraffins, diesel fluids, mineral oils, desulfurized hydrogenated kerosenes, and any combination thereof.

Examples of aqueous fluids suitable for use as the discontinuous aqueous phase may include, but are not limited to, fresh water, saltwater (e.g., water containing one or more salts dissolved therein), brine (e.g., saturated salt water), seawater, and any combination thereof. The pH of the discontinuous aqueous phase may, in some instances, range from a lower limit of about 0, 1, 2, 3, 4, 5, 6, 7, 8, or 9 to an upper limit of about 11, 10, 9, 8, 7, or 6, wherein the pH may range from any lower limit to any upper limit and encompass any subset therebetween. For example in carbonate subterranean formations, the pH of the discontinuous aqueous phase may be about pH 7 to about pH 11, about pH 8 to about pH 11, or about pH 9 to about pH 11 to mitigate formation damage when the emulsion is broken.

Suitable invert emulsions may have an oil-to-water volume ratio from a lower limit of greater than about 50:50, 55:45, 60:40, 65:35, 70:30, 75:25, or 80:20 to an upper limit of less than about 100:0, 95:5, 90:10, 85:15, 80:20, 75:25, 70:30, or 65:35, wherein the amount may range from any lower limit to any upper limit and encompass any subset therebetween.

Examples of emulsifiers suitable for use in the treatment fluids described herein may include, but are not limited to, a sulfate (e.g., ammonium aluryl sulfate, sodium lauryl sulfate, sodium laureth sulfate, or sodium myreth sulfate), a sulfonate (e.g., perfluorooctanesulfonate, or perfluorobutanesulfonate, linear (C₁-C₁₀)alkylbenzene sulfonate), a phosphate, a carboxylate, dioctyl sodium sulfosuccinate, sodium stearate, sodium lauroyl sarcosinate, perfluorononanoate, perfluorooctanoate, octenidine dihydrochloride, cetyl trimethylammonium bromide, cetyl trimethylammonium chloride, cetylpyridinium chloride, benzalkonium chloride, benzethonium chloride, 5-bromo-5-nitro-1,3-dioxane, dimethyldiactadecylammonium chloride, cetrimonium bromide, dioctadecyldimethylammonium bromide, 3-[(3-cholamidopropyl)dimethylammonio]-1-propanesulfonate, cocamidopropyl hydroxysultaine, cocamidopropyl betaine, lecithin, tri(C₁-C₁₀)alkylammonium halide, substituted or unsubstituted fatty alcohol, substituted or unsubstituted fatty acid (e.g., polyaminated (C₃-C₅₀)fatty acid), substituted or unsubstituted fatty acid ester (e.g., polyaminated (C₃-C₅₀)fatty acid (C₁-C₁₀)alkyl ester), a substituted or unsubstituted poly((C₁-C₁₀)hydrocarbylene oxide) independently having H or (C₁-C₁₀)hydrocarbylene as end-groups, a polyoxyethylene glycol alkyl ether (e.g., octaethylene glycol monododecyl ether, pentaethylene glycol monododecyl ether), a polyoxypropylene glycol ether, a glucoside alkyl ether (e.g., decyl glucoside, lauryl glucoside, octyl glucoside), a polyoxyethylene glycol octylphenol ether (e.g., TRITON X-100), a polyoxyethylene glycol alkylphenol ether (e.g., nonoxynol-9), a glycerol alkyl ether (e.g., glyceryl laurate), a polyoxyethylene glycol sorbitan alkyl ester (e.g., monopalmitate, monosterate, monooleate, or a polysorbate, such as polyoxyethylene (20) sorbitan monolaurate), cocamide monoethanolamine, cocamide diethanolamine, dodecyldimethylaminde oxide, a poloxamer, a polyethoxylated tallow amine, a carboxylic acid-terminated polyamide (e.g., having fatty (C₁₀-C₅₀)hydrocarbyl units between the amide units), a substituted or unsubstituted (C₂-C₅₀)hydrocarbyl-carboxylic acid or a (C₁-C₅₀)hydrocarbyl ester thereof, a mono- or poly-(substituted or unsubstituted (C₂-C₁₀)alkylene) diol having 0, 1, or 2 hydroxy groups etherified with a (C₁-C₅₀)hydrocarbyl group (wherein each (C₁₀-C₅₀)hydrocarbyl and (C₁-C₅₀)hydrocarbyl is independently selected and is independently substituted or unsubstituted, and wherein each (C₁₀-C₅₀)hydrocarbyl is independently interrupted by 0, 1, 2, or 3 groups selected from —O—, —S—, and substituted or unsubstituted —NH—), a mono- or poly-(C₂-C₁₀)alkylene diol mono(C₁-C₁₀)alkyl ether, a (C₂-C₃₀)alkanoic acid, and a (C₂-C₃₀)alkenoic acid, ethylene glycol monobutyl ether, diethylene glycol monobutyl ether, a (C₄-C₅₀) alpha-olefin, an isomerized (C₄-C₅₀) alpha-olefin, ethylene glycol, propylene glycol, petroleum distillate, hydrotreated petroleum distillate, diesel, naphthalene, and the like, and any combination thereof. Examples of commercially available emulsifiers include, but are not limited to, LE SUPERMUL™ (an invert emulsifier, available from Halliburton Energy Services, Inc.), FORTI-MUL™ (a primary emulsifier for invert emulsions, available from Halliburton Energy Services, Inc.), EZ MUL® NT (a secondary emulsifier for invert emulsions, available from Halliburton Energy Services, Inc.), AF-70 (an emulsifier, available from Halliburton Energy Services, Inc.), and AF-61 (an emulsifier, available from Halliburton Energy Services, Inc.).

In some instances when neutral or basic discontinuous aqueous phases are used (e.g., in conjunction with carbonation formations), preferred emulsifiers may include, but are not limited to, a mixture of ethylene glycol monobutyl ether, and diethylene glycol monobutyl ether; a mixture of hydrotreated light petroleum distillate, ethylene glycol monobutyl ether, and diethylene glycol monobutyl ether; and the like.

The emulsifier may be present in a treatment fluid described herein in an amount range from a lower limit of about 0.01%, 0.1%, 0.5%, or 1% by weight of the treatment fluid to an upper limit of about 10%, 5%, or 2% by weight of the treatment fluid, wherein the amount of emulsifier may range from any lower limit to any upper limit and encompass any subset therebetween.

Examples of water-swellable polymers suitable for use in the treatment fluids described herein may include, but are not limited to, crosslinked polyacrylamide, crosslinked polyacrylate, crosslinked hydrolyzed polyacrylonitrile, salts of carboxyalkyl starch, salts of carboxymethyl starch, salts of carboxyalkyl cellulose, hydroxylethyl cellulose, salts of crosslinked carboxyalkyl polysaccharide, crosslinked copolymers of acrylamide and acrylate monomers, starch grafted with acrylonitrile and acrylate monomers, crosslinked polymers of two or more of allylsulfonates, 2-acrylamido-2-methyl-1-propanesulfonic acid, 3-allyloxy-2-hydroxy-1-propane-sulfonic acid, acrylamide, acrylic acid monomers, and any combination thereof. Examples of commercially available water-swellable polymers include, but are not limited to, CRYSTALSEAL® (a water-swellable, synthetic polymer, available from Halliburton Energy Services, Inc.), DIAMOND SEAL@ (a water-swellable, synthetic polymer, available from Halliburton Energy Services, Inc.), and AD-200 (a water-swellable, synthetic polymer, available from Hychem, Inc.).

In some embodiments, an unswollen water-swellable polymer may have a particle size that may range from a lower limit of about 100 mesh (US Standard Mesh Size), 80 mesh, or 50 mesh to an upper limit of about 6 mesh, 10 mesh, or 20 mesh, and wherein the particle size may range from any lower limit to any upper limit and encompasses any subset therebetween. In some embodiments, an unswollen water-swellable polymer may have a size in at least one dimension (e.g., width, length, or diameter) ranging from a lower limit of about 500 microns or 1 mm to an upper limit of about 4 mm or 2 mm, and wherein the size in at least one dimension may range from any lower limit to any upper limit and encompasses any subset therebetween. Particles of the unswollen water-swellable polymer may be in any shape including, but not limited to, cubic, spherical, elongate (e.g., rods or fibers), flakes, rhomboidal, ellipsoidal, any hybrid thereof, and any combination thereof.

The water-swellable polymer may be present in a treatment fluid described herein in an amount range from a lower limit of about 0.001%, 0.01%, 0.1%, or 1% by weight of the treatment fluid to an upper limit of about 25%, 20%, 10%, or 5% by weight of the treatment fluid (as measured by the unswollen weight of the water-swellable polymer), wherein the amount of water-swellable polymer may range from any lower limit to any upper limit and encompass any subset therebetween. The concentration of water-swellable polymer in the treatment fluid may depend on many factors including, but not limited to, the pumping and placement time (e.g., lower concentrations may be used for longer pumping and placement times so as to allow sufficient time to place the water-swellable polymer before swelling blocks fluid flow) and the maximum amount of swelling the water-swellable polymer is capable of (e.g., water-swellable polymer with greater maximum swelling may be used at lower concentrations so as to allow sufficient time to place the water-swellable polymer before swelling blocks fluid flow).

In some instance, the treatment fluids described herein may optionally further comprise additives, for example, salts, weighting agents, inert solids, fluid loss control agents, corrosion inhibitors, emulsion thinners, emulsion thickeners, viscosifying agents, pH control additives, emulsion breakers, stabilizers, friction reducers, clay stabilizing agents, and the like, and any combination thereof.

For example, the discontinuous aqueous phase may include pH control additives to maintain a desired pH of the aqueous fluid. In another example, the treatment fluid may include a breaker that breaks the emulsion, so that the discontinuous aqueous phase and water-swellable polymer contact. In yet another example, the treatment fluid may include a breaker at a low concentration that in combination with the wellbore conditions breaks the emulsion, so that the discontinuous aqueous phase and water-swellable polymer contact. As described further herein, in some instances, the wellbore conditions may be sufficient to break the emulsion with no breaker in the treatment fluid.

The treatment fluids described herein may be useful in a variety of wellbore operations where reduction of fluid flow through a portion of a subterranean formation are desired. For example, a treatment fluid (e.g., comprising an emulsion with an continuous oil phase and a discontinuous aqueous phase, an emulsifier, a water-swellable polymer suspended in the continuous oil phase, and optionally additives, wherein the aqueous discontinuous phase has a pH of about 0 to about 11) may be introduced into a wellbore penetrating a subterranean formation. Then, after placement in a portion of a subterranean formation, the emulsion may be broken, thereby allowing the water-swellable polymer to swell to a greater volume (i.e., for a swollen polymer) in situ and reduce the fluid flow through the portion of the subterranean formation.

In some instances, the portion of the subterranean formation in which fluid flow is reduced may be considered far wellbore. As used herein, the term “far wellbore” refers to about 300 ft to about 1200 ft from the wellbore.

In some embodiments, the temperature of the portion of the subterranean formation may be sufficiently high (e.g., about 200° F. or greater) to cause the emulsion to break. In such instances, breakers may optionally be included in the treatment fluid.

After in situ swelling of the water-swellable polymer, subsequent operations may be performed. In some instances, the reduced fluid flow may mitigate or prevent the flow of water from the formation to the wellbore, which may increase the ratio of produced hydrocarbon to produced water, thereby increasing the efficacy of production operations.

In some embodiments, another treatment fluid may be introduced into the wellbore after in situ swelling of the water-swellable polymer. In some instances, the reduced fluid flow through the portion of the subterranean formation may divert a portion of the subsequent treatment fluid to another portion of the subterranean formation.

In some embodiments, a sweeping fluid in an enhanced oil recovery (EOR) flooding operation may be introduced into the subterranean formation after in situ swelling of the water-swellable polymer in the subterranean formation. The sweeping fluid may be diverted to sweep oil from unswept zones by preventing the loss of fluid into high permeability zones now at least partially plugged by the swollen polymer.

In various embodiments, systems configured for delivering the treatment fluids described herein to a downhole location are described. In various embodiments, the systems can comprise a pump fluidly coupled to a tubular that penetrates a wellbore penetrating a subterranean formation, the tubular containing a treatment fluid described herein (e.g., comprising an emulsion with an continuous oil phase and a discontinuous aqueous phase, an emulsifier, a water-swellable polymer suspended in the continuous oil phase, and optionally additives, wherein the aqueous discontinuous phase has a pH of about 0 to about 11).

The pump may be a high pressure pump in some embodiments. As used herein, the term “high pressure pump” will refer to a pump that is capable of delivering a fluid downhole at a pressure of about 1000 psi or greater. A high pressure pump may be used when it is desired to introduce the treatment fluid to a subterranean formation at or above a fracture gradient of the subterranean formation, but it may also be used in cases where fracturing is not desired. In some embodiments, the high pressure pump may be capable of fluidly conveying particulate matter, such as proppant particulates, into the subterranean formation. Suitable high pressure pumps will be known to one having ordinary skill in the art and may include, but are not limited to, floating piston pumps and positive displacement pumps.

In other embodiments, the pump may be a low pressure pump. As used herein, the term “low pressure pump” will refer to a pump that operates at a pressure of about 1000 psi or less. In some embodiments, a low pressure pump may be fluidly coupled to a high pressure pump that is fluidly coupled to the tubular. That is, in such embodiments, the low pressure pump may be configured to convey the treatment fluid to the high pressure pump. In such embodiments, the low pressure pump may “step up” the pressure of the treatment fluid before it reaches the high pressure pump.

In some embodiments, the systems described herein can further comprise a mixing tank that is upstream of the pump and in which the treatment fluid is formulated. In various embodiments, the pump (e.g., a low pressure pump, a high pressure pump, or a combination thereof) may convey the treatment fluid from the mixing tank or other source of the treatment fluid to the tubular. In other embodiments, however, the treatment fluid can be formulated offsite and transported to a worksite, in which case the treatment fluid may be introduced to the tubular via the pump directly from its shipping container (e.g., a truck, a railcar, a barge, or the like) or from a transport pipeline. In either case, the treatment fluid may be drawn into the pump, elevated to an appropriate pressure, and then introduced into the tubular for delivery downhole.

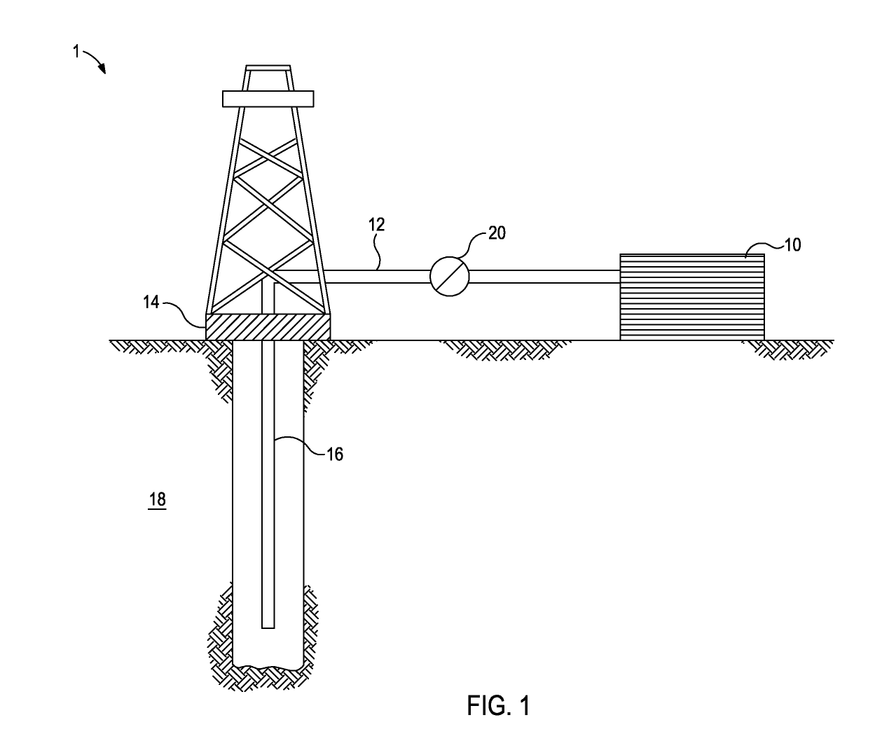

FIG. 1 shows an illustrative schematic of a system that can deliver treatment fluids of the present disclosure to a downhole location, according to one or more embodiments. It should be noted that while FIG. 1 generally depicts a land-based system, it is to be recognized that like systems may be operated in subsea locations as well. As depicted in FIG. 1, system 1 may include mixing tank 10, in which a treatment fluid of the present disclosure may be formulated. The treatment fluid may be conveyed via line 12 to wellhead 14, where the treatment fluid enters tubular 16, tubular 16 extending from wellhead 14 into subterranean formation 18. Upon being ejected from tubular 16, the treatment fluid may subsequently penetrate into subterranean formation 18. In some instances, tubular 16 may have a plurality of orifices (not shown) through which the treatment fluid of the present disclosure may enter the wellbore proximal to a portion of the subterranean formation 18 to be treated. In some instances, the wellbore may further comprise equipment or tools (not shown) for zonal isolation of a portion of the subterranean formation 18 to be treated.

Pump 20 may be configured to raise the pressure of the treatment fluid to a desired degree before its introduction into tubular 16. It is to be recognized that system 1 is merely exemplary in nature and various additional components may be present that have not necessarily been depicted in FIG. 1 in the interest of clarity. Non-limiting additional components that may be present include, but are not limited to, supply hoppers, valves, condensers, adapters, joints, gauges, sensors, compressors, pressure controllers, pressure sensors, flow rate controllers, flow rate sensors, temperature sensors, and the like.

Although not depicted in FIG. 1, the treatment fluid may, in some embodiments, flow back to wellhead 14 and exit subterranean formation 18. In some embodiments, the treatment fluid that has flowed back to wellhead 14 may subsequently be recovered and recirculated to subterranean formation 18.

It is also to be recognized that the disclosed treatment fluids may also directly or indirectly affect the various downhole equipment and tools that may come into contact with the treatment fluids during operation. Such equipment and tools may include, but are not limited to, wellbore casing, wellbore liner, completion string, insert strings, drill string, coiled tubing, slickline, wireline, drill pipe, drill collars, mud motors, downhole motors and/or pumps, surface-mounted motors and/or pumps, centralizers, turbolizers, scratchers, floats (e.g., shoes, collars, valves, etc.), logging tools and related telemetry equipment, actuators (e.g., electromechanical devices, hydromechanical devices, etc.), sliding sleeves, production sleeves, plugs, screens, filters, flow control devices (e.g., inflow control devices, autonomous inflow control devices, outflow control devices, etc.), couplings (e.g., electro-hydraulic wet connect, dry connect, inductive coupler, etc.), control lines (e.g., electrical, fiber optic, hydraulic, etc.), surveillance lines, drill bits and reamers, sensors or distributed sensors, downhole heat exchangers, valves and corresponding actuation devices, tool seals, packers, cement plugs, bridge plugs, and other wellbore isolation devices, or components, and the like. Any of these components may be included in the systems generally described above and depicted in FIG. 1.

Embodiments disclosed herein include:

Embodiment A—introducing a treatment fluid into a wellbore penetrating a subterranean formation, the treatment fluid comprising an emulsion with an continuous oil phase and a discontinuous aqueous phase, an emulsifier, and a water-swellable polymer suspended in the continuous oil phase, wherein the aqueous discontinuous phase has a pH of about 0 to about 11; breaking the emulsion while the treatment fluid in a portion of the subterranean formation; and swelling the water-swellable polymer to a swollen polymer, thereby reducing fluid flow through the portion of the subterranean formation;

Embodiment B—introducing a treatment fluid into a wellbore penetrating a carbonate subterranean formation, the treatment fluid comprising an emulsion with an continuous oil phase and a discontinuous aqueous phase, an emulsifier; and a water-swellable polymer suspended in the continuous oil phase, wherein the aqueous discontinuous phase has a pH of about 7 to about 11; breaking the emulsion while the treatment fluid in a portion of the subterranean formation; and swelling the water-swellable polymer to a swollen polymer, thereby reducing fluid flow through the portion of the subterranean formation; and

Embodiment C—a tubular extending from a wellhead and into a wellbore penetrating a subterranean formation (e.g., a carbonate subterranean formation); and a pump fluidly coupled to a tubular, the tubular containing a treatment fluid that comprises an emulsion with an continuous oil phase and a discontinuous aqueous phase, an emulsifier, and a water-swellable polymer suspended in the continuous oil phase, wherein the aqueous discontinuous phase has a pH of about 0 to about 11.

Each of embodiments A, B, and C may have one or more of the following additional elements in any combination: Element 1: wherein the emulsifier is present in an amount of about 0.01% to about 10% by weight of the treatment fluid; Element 2: wherein the water-swellable polymer is present in an amount of about 0.001% to about 25% by weight of the treatment fluid; Element 3: wherein the discontinuous aqueous phase comprises seawater; Element 4: wherein the emulsifier comprises at least one selected from the group consisting of: a sulfate (e.g., ammonium aluryl sulfate, sodium lauryl sulfate, sodium laureth sulfate, or sodium myreth sulfate), a sulfonate (e.g., perfluorooctanesulfonate, or perfluorobutanesulfonate, linear (C₁-C₁₀)alkylbenzene sulfonate), a phosphate, a carboxylate, dioctyl sodium sulfosuccinate, sodium stearate, sodium lauroyl sarcosinate, perfluorononanoate, perfluorooctanoate, octenidine dihydrochloride, cetyl trimethylammonium bromide, cetyl trimethylammonium chloride, cetylpyridinium chloride, benzalkonium chloride, benzethonium chloride, 5-bromo-5-nitro-1,3-dioxane, dimethyldiactadecylammonium chloride, cetrimonium bromide, dioctadecyldimethylammonium bromide, 3-[(3-cholamidopropyl)dimethylammonio]-1-propanesulfonate, cocamidopropyl hydroxysultaine, cocamidopropyl betaine, lecithin, tri(C₁-C₁₀)alkylammonium halide, substituted or unsubstituted fatty alcohol, substituted or unsubstituted fatty acid (e.g., polyaminated (C₃-C₅₀)fatty acid), substituted or unsubstituted fatty acid ester (e.g., polyaminated (C₃-C₅₀)fatty acid (C₁-C₁₀)alkyl ester), a substituted or unsubstituted poly((C₁-C₁₀)hydrocarbylene oxide) independently having H or (C₁-C₁₀)hydrocarbylene as end-groups, a polyoxyethylene glycol alkyl ether (e.g., octaethylene glycol monododecyl ether, pentaethylene glycol monododecyl ether), a polyoxypropylene glycol ether, a glucoside alkyl ether (e.g., decyl glucoside, lauryl glucoside, octyl glucoside), a polyoxyethylene glycol octylphenol ether (e.g., TRITON X-100), a polyoxyethylene glycol alkylphenol ether (e.g., nonoxynol-9), a glycerol alkyl ether (e.g., glyceryl laurate), a polyoxyethylene glycol sorbitan alkyl ester (e.g., monopalmitate, monosterate, monooleate, or a polysorbate, such as polyoxyethylene (20) sorbitan monolaurate), cocamide monoethanolamine, cocamide diethanolamine, dodecyldimethylaminde oxide, a poloxamer, a polyethoxylated tallow amine, a carboxylic acid-terminated polyamide (e.g., having fatty (C₁₀-C₅₀)hydrocarbyl units between the amide units), a substituted or unsubstituted (C₂-C₅₀)hydrocarbyl-carboxylic acid or a (C₁-C₅₀)hydrocarbyl ester thereof, a mono- or poly-(substituted or unsubstituted (C₂-C₁₀)alkylene) diol having 0, 1, or 2 hydroxy groups etherified with a (C₁-C₅₀)hydrocarbyl group (wherein each (C₁₀-C₅₀)hydrocarbyl and (C₁-C₅₀)hydrocarbyl is independently selected and is independently substituted or unsubstituted, and wherein each (C₁₀-C₅₀)hydrocarbyl is independently interrupted by 0, 1, 2, or 3 groups selected from —O—, —S—, and substituted or unsubstituted —NH—), a mono- or poly-(C₂-C₁₀)alkylene diol mono(C₁-C₁₀)alkyl ether, a (C₂-C₃₀)alkanoic acid, and a (C₂-C₃₀)alkenoic acid, ethylene glycol monobutyl ether, diethylene glycol monobutyl ether, a (C₄-C₅₀) alpha-olefin, an isomerized (C₄-C₅₀) alpha-olefin, ethylene glycol, propylene glycol, petroleum distillate, hydrotreated petroleum distillate, diesel, naphthalene, and the like, and any combination thereof; Element 5: wherein the pH is about 7 to about 11; Element 6: Element 5 and wherein the emulsifier comprises at least one selected from the group consisting of: a mixture of ethylene glycol monobutyl ether, and diethylene glycol monobutyl ether and a mixture of hydrotreated light petroleum distillate, ethylene glycol monobutyl ether, and diethylene glycol monobutyl ether; Element 7: wherein the treatment fluid further comprises a breaker; and Element 8: wherein the portion of the subterranean formation (or the carbonate subterranean formation) is about 200° F. or greater.

By way of non-limiting example, exemplary combinations applicable to A, B, C include: Element 1 in combination with Element 2 and optionally Element 3; Element 2 in combination with Element 3; Element 4 in combination with at least one of Elements 1-3; Element 5 and optionally Element 6 in combination with at least one of Elements 1-3; and at least one of Elements 7-8 in combination with at least one of Elements 1-6 including any of the foregoing combinations.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments described herein. At the very least, and not as an attempt to limit the application of the doctrine of equivalents to the scope of the claim, each numerical parameter should at least be construed in light of the number of reported significant digits and by applying ordinary rounding techniques. Further, when a series of possible upper and lower limits are provided where the term “about” is provided only at the beginning of the list, the term “about” modifies each of the values of the list. Further, when a series of possible upper and lower limits are provided, one skilled in the art would recognize that the upper limit should be chose to be greater than the lower limit.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill the art and having benefit of this disclosure.

While compositions and methods are described herein in terms of “comprising” various components or steps, the compositions and methods can also “consist essentially of” or “consist of” the various components and steps.

To facilitate a better understanding of the embodiments of the present invention, the following examples of preferred or representative embodiments are given. In no way should the following examples be read to limit, or to define, the scope of the invention.

### EXAMPLES

### Example 1

Three invert emulsions were prepared with discontinuous aqueous phases of pH 0, 7, and 11 and EZ MUL® NT emulsifier. No separation was observed for any of the invert emulsions after three days at room temperature. Then, each sample was paced in a 180° F. water bath for about 12 hours with no visible separation, color changes, or viscosity changes.

### Example 2

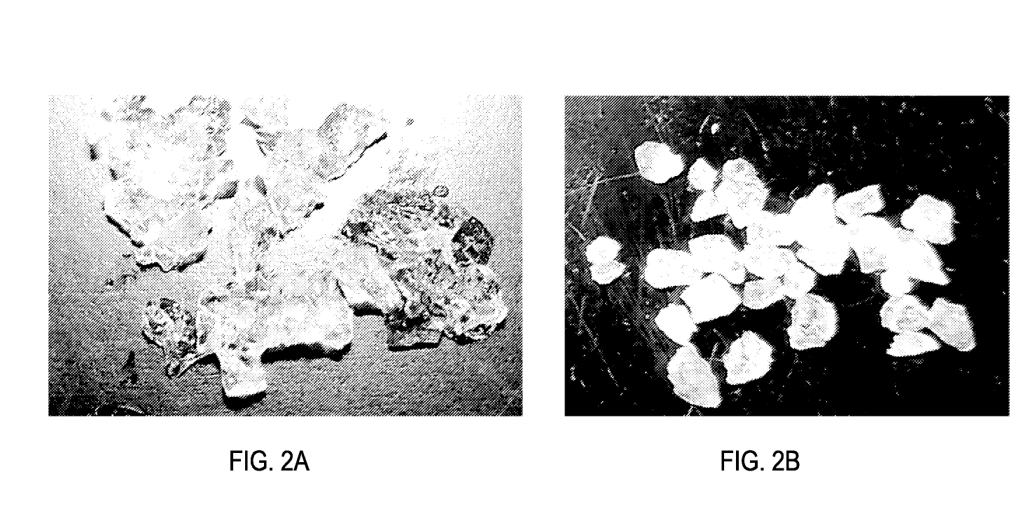

An invert emulsion was prepared with a seawater discontinuous aqueous phase (pH-8), EZ MUL® NT emulsifier, and CRYSTALSEAL® water-swellable polymer. As a control sample, CRYSTALSEAL® water-swellable polymer was placed in seawater. After about 5 minutes, the CRYSTALSEAL® was removed from the two samples. The CRYSTALSEAL® in the control samples was significantly swollen from hydration (FIG. 2A) while the CRYSTALSEAL® in the invert emulsion was still substantially unchanged (FIG. 2B).

### Example 3

An invert emulsion was prepared with a seawater discontinuous aqueous phase (pH-8), EZ MUL® NT emulsifier, and CRYSTALSEAL® water-swellable polymer. The sample was placed in a 180° F. water bath for about 4 hours. Upon visual inspection, the CRYSTALSEAL® had swollen to size indicating nearly full hydration.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

The invention claimed is:

1. A method comprising:
introducing a treatment fluid into a wellbore penetrating a subterranean formation, the treatment fluid comprising an emulsion with an continuous oil phase and a discontinuous aqueous phase, an emulsifier, and a water-swellable polymer suspended in the continuous oil phase, wherein the aqueous discontinuous phase has a pH of about 0 to about 11;
breaking the emulsion while the treatment fluid in a portion of the subterranean formation; and
swelling the water-swellable polymer to a swollen polymer, thereby reducing fluid flow through the portion of the subterranean formation.

2. The method of claim 1, wherein the emulsifier is present in an amount of about 0.01% to about 10% by weight of the treatment fluid.

3. The method of claim 1, wherein the water-swellable polymer is present in an amount of about 0.001% to about 25% by weight of the treatment fluid.

4. The method of claim 1, wherein the discontinuous aqueous phase comprises seawater.

5. The method of claim 1, wherein the treatment fluid further comprises a breaker.

6. The method of claim 1, wherein the portion of the subterranean formation is about 200° F. or greater.

7. A method comprising:
introducing a treatment fluid into a wellbore penetrating a carbonate subterranean formation, the treatment fluid comprising an emulsion with an continuous oil phase and a discontinuous aqueous phase, an emulsifier;
and a water-swellable polymer suspended in the continuous oil phase, wherein the aqueous discontinuous phase has a pH of about 7 to about 11;
breaking the emulsion while the treatment fluid in a portion of the subterranean formation; and
swelling the water-swellable polymer to a swollen polymer, thereby reducing fluid flow through the portion of the subterranean formation.

8. The method of claim 7, wherein the emulsifier is present in an amount of about 0.01% to about 10% by weight of the treatment fluid.

9. The method of claim 7, wherein the emulsifier comprises at least one selected from the group consisting of: a mixture of ethylene glycol monobutyl ether, and diethylene glycol monobutyl ether and a mixture of hydrotreated light petroleum distillate, ethylene glycol monobutyl ether, and diethylene glycol monobutyl ether.

10. The method of claim 7, wherein the water-swellable polymer is present in an amount of about 0.001% to about 25% by weight of the treatment fluid.

11. The method of claim 7, wherein the discontinuous aqueous phase comprises seawater.

12. The method of claim 7, wherein the treatment fluid further comprises a breaker.

13. The method of claim 7, wherein the portion of the subterranean formation is about 200° F. or greater.

14. A system comprising:
a tubular extending from a wellhead and into a wellbore penetrating a subterranean formation; and
a pump fluidly coupled to a tubular, the tubular containing a treatment fluid that comprises an emulsion with an continuous oil phase and a discontinuous aqueous phase, an emulsifier, and a water-swellable polymer suspended in the continuous oil phase, wherein the aqueous discontinuous phase has a pH of about 0 to about 11.

15. The system of claim 14, wherein the pH is about 7 to about 11.

16. The system of claim 15, wherein the emulsifier comprises at least one selected from the group consisting of: a mixture of ethylene glycol monobutyl ether, and diethylene glycol monobutyl ether and a mixture of hydrotreated light petroleum distillate, ethylene glycol monobutyl ether, and diethylene glycol monobutyl ether.

17. The system of claim 14, wherein the emulsifier is present in an amount of about 0.01% to about 10% by weight of the treatment fluid.

18. The system of claim 14, wherein the water-swellable polymer is present in an amount of about 0.001% to about 25% by weight of the treatment fluid.

19. The system of claim 14, wherein the discontinuous aqueous phase comprises seawater.

20. The system of claim 14, wherein the subterranean formation is a carbonate subterranean formation.
